(19) United States
(12) Patent Application Publication
(10) Pub. No.: US 2018/0020961 A1
(43) Pub. Date: Jan. 25, 2018

(54) MEASUREMENT DEVICE AND DETECTION DEVICE

(71) Applicant: SEIKO EPSON CORPORATION, Tokyo (JP)

(72) Inventor: Tetsuo MANO, Fujimi-machi (JP)

(73) Assignee: SEIKO EPSON CORPORATION, Tokyo (JP)

(21) Appl. No.: 15/639,388

(22) Filed: Jun. 30, 2017

(30) Foreign Application Priority Data
Jul. 19, 2016 (JP) 2016-141677

(51) Int. Cl. A61B 5/1455; A61B 5/01; A61B 5/00

(52) U.S. Cl. CPC A61B 5/14552; A61B 5/6844; A61B 5/01; A61B 5/681; A61B 2560/0242

(57) ABSTRACT

A measurement device includes a first light emitting unit that emits light with a first wavelength onto a measurement site, a second light emitting unit that emits light with a second wavelength different from the first wavelength onto the measurement site, a light receiving unit that receives light which passes through an inside of the measurement site and generates a detection signal, and an analysis processing unit that calculates a degree of oxygen saturation from the detection signal. Distances between light emission positions by each of the first light emitting unit and the second light emitting unit and a light reception position by the light receiving unit are variable.

### BACKGROUND

### 1. Technical Field

The present invention relates to a technology for measuring biological information such as a degree of oxygen saturation.

### 2. Related Art

Various measurement techniques for noninvasively measuring biological information such as a degree of oxygen saturation are proposed in the related art. For example, JP-T-2013-533774 discloses a configuration in which a light receiving element receives light which is emitted from a light emitting element that passes through a living body thereby measuring the degree of oxygen saturation. A distance between the light emitting element and the light receiving element is selected such that desired measurement accuracy is realized. It is possible to estimate the degree of oxygen saturation according to a signal component ratio between normal components and variation components (pulsation components) in a detection signal indicating a light reception level from a measurement object.

However, a state of the living body which becomes the measurement object is frequently changed. For example, it may enter a low perfusion state in which a blood flow rate is decreased, in a low temperature environment. Since the signal component ratio of the detection signal decreases in the low perfusion state, there is a problem that the measurement accuracy of the degree of oxygen saturation decreases.

### SUMMARY

An advantage of some aspects of the invention is to specify a degree of oxygen saturation with high accuracy even in a case where a state of a measurement object changes.

A measurement device according to a preferred aspect of the invention includes a first light emitting unit that emits light with a first wavelength onto a measurement site, a second light emitting unit that emits light with a second wavelength different from the first wavelength onto the measurement site, a light receiving unit that receives light which passes through an inside of the measurement site and generates a detection signal, and an analysis processing unit that calculates a degree of oxygen saturation from the detection signal, in which distances between light emission positions by each of the first light emitting unit and the second light emitting unit and a light reception position by the light receiving unit are variable. In this configuration, since the distances between the light emission positions by each of the first light emitting unit and the second light emitting unit and the light reception position by the light receiving unit are variable, it is possible to specify the degree of oxygen saturation with high accuracy, even in a case where a state of the measurement site changes (for example, a low perfusion state).

In the preferable aspect of the invention, the analysis processing unit may include a first processing unit that calculates a signal component ratio between normal components and variation components of the detection signal, and a second processing unit that specifies the degree of oxygen saturation from the signal component ratio. In the aspect described above, it is possible to specify a degree of oxygen saturation with high accuracy by using a signal component ratio between normal components and variation components in a detection signal.

The measurement device according to the preferable aspect of the invention may include a measurement control unit that increases a distance between a light emission position and a light reception position, in a case where a signal component ratio is less than a first threshold. In the aspect described above, since a distance between a light emission position and a light reception position increases in a case where a signal component ratio is less than a first threshold, it is possible to measure a degree of oxygen saturation with high accuracy, for example, even in a low perfusion state. In addition, it is possible to reduce power necessary for making a first light emitting unit and a second light emitting unit emit light, according to a configuration in which a distance between a light emission position and a light reception position decreases, in a case where a signal component ratio exceeds a second threshold exceeding a first threshold.

In the preferable aspect of the invention, each of a first light emitting unit and a second light emitting unit may include a plurality of light emitting elements whose distances from the light receiving unit are different from each other, and the measurement control unit changes a distance between the light emission position and the light reception position by selectively making any one of the plurality of light emitting elements emit light, with respect to each of the first light emitting unit and the second light emitting unit. In the aspect described above, it is possible to change a distance between a light emission position and a light reception position by a simple configuration in which any one of the plurality of light emitting elements selectively emits light.

In the preferable aspect of the invention, the light receiving unit may include a plurality of light receiving elements whose distances from the first light emitting unit and the second light emitting unit are different from each other, and the measurement control unit may change a distance between the light emission position and the light reception position by selecting any one of the plurality of light receiving elements. In the aspect described above, it is possible to change a distance between a light emission position and a light reception position by a simple configuration in which any one of the plurality of light receiving elements is selected. An operation of selecting any one of the plurality of light receiving elements is, for example, an operation of selecting a light receiving element which performs generation of a detection signal from the plurality of light receiving elements, or an operation of selecting any one of detection signals which are generated by the plurality of light receiving elements.

In the preferable aspect of the invention, the distance between the light emission position and the light reception position may be changeable by an operation of a user, and the measurement device may further include an operation instruction unit that notifies a user of instruction to increase the distance between the light emission position and the light reception position, in a case where the signal component ratio is less than a first threshold. In the aspect described above, since the user is notified of instruction to increase the distance between the light emission position and the light reception position, in a case where the signal component ratio is less than a first threshold, it is possible to measure a degree of oxygen saturation with high accuracy even in a low perfusion state. In addition, it is possible to reduce power necessary for making a first light emitting unit and a second light emitting unit emit light, according to a configuration in which the user is notified of instruction to decrease a distance between a light emission position and a light reception position, in a case where a signal component ratio exceeds a second threshold exceeding a first threshold.

The measurement device according to the preferable aspect of the invention may include a temperature detection unit that detects a skin temperature or an ambient temperature of a measurement site as a reference temperature, and a measurement control unit that increases the distance between the light emission position and the light reception position, in a case where the reference temperature is lower than a threshold. In the aspect described above, since the distance between the light emission position and the light reception position increases, in a case where the reference temperature is lower than a threshold, it is possible to measure a degree of oxygen saturation with high accuracy, for example, even in a low perfusion state. In addition, the measurement device according to another aspect of the invention includes a temperature detection unit that detects a skin temperature or an ambient temperature of the measurement site as a reference temperature, and an operation instruction unit that notifies a user of instruction to increase the distance between the light emission position and the light reception position, in a case where the reference temperature is lower than a threshold. In the above aspect, since the user is notified of the instruction to increase the distance between the light emission position and the light reception position, in a case where the reference temperature is lower than a threshold, it is possible to measure a degree of oxygen saturation with high accuracy, even in a low perfusion state.

A detection device according to a preferable aspect of the invention generates a detection signal which is used for measuring a degree of oxygen saturation, and includes a first light emitting unit that emits light with a first wavelength onto a measurement site, a second light emitting unit that emits light with a second wavelength different from the first wavelength onto the measurement site, and a light receiving unit that receives light which passes through an inside of the measurement site and generates a detection signal, in which distances between light emission positions by each of the first light emitting unit and the second light emitting unit and a light reception position by the light receiving unit are variable. In the aspect described above, since the distances between the light emission positions by each of the first light emitting unit and the second light emitting unit and the light reception position by the light receiving unit are variable, it is possible to generate the detection signal which may specify the degree of oxygen saturation with high accuracy, even in a case where a state of the measurement site changes (for example, a low perfusion state).

### BRIEF DESCRIPTION OF THE DRAWINGS

The invention will be described with reference to the accompanying drawings, wherein like numbers reference like elements.

FIG. 1 is a side view of a measurement device according to a first embodiment of the invention.

FIG. 2 is a configuration diagram focusing on a function of the measurement device.

FIG. 3 is a plan view of a detection device.

FIG. 4 is a configuration diagram of an analysis processing unit.

FIG. 5 is a graph illustrating a relationship between signal component ratios and a measurement error.

FIG. 6 is a graph illustrating relationships between a distance between emission and reception of light and the signal component ratios.

FIG. 7 is a graph illustrating a relationship between a skin temperature and the signal component ratio.

FIG. 8 is a flowchart of processing in which a measurement control unit sets the distance between emission and reception of light.

FIG. 9 is a graph illustrating a relationship between a degree of oxygen saturation and a variation ratio.

FIG. 10 is a plan view of a detection device according to a second embodiment.

FIG. 11 is a plan view and a sectional view of a detection device according to a third embodiment.

FIG. 12 is a functional configuration diagram of a measurement device according to the third embodiment.

FIG. 13 is a flowchart of processing in which an operation instruction unit notifies a user of a change instruction.

FIG. 14 is a plan view and a sectional view of a detection device according to a fourth embodiment.

FIG. 15 is a configuration diagram of a measurement device according to a fifth embodiment.

FIG. 16 is a configuration diagram of a measurement device according to a sixth embodiment.

### DESCRIPTION OF EXEMPLARY EMBODIMENTS

### First Embodiment

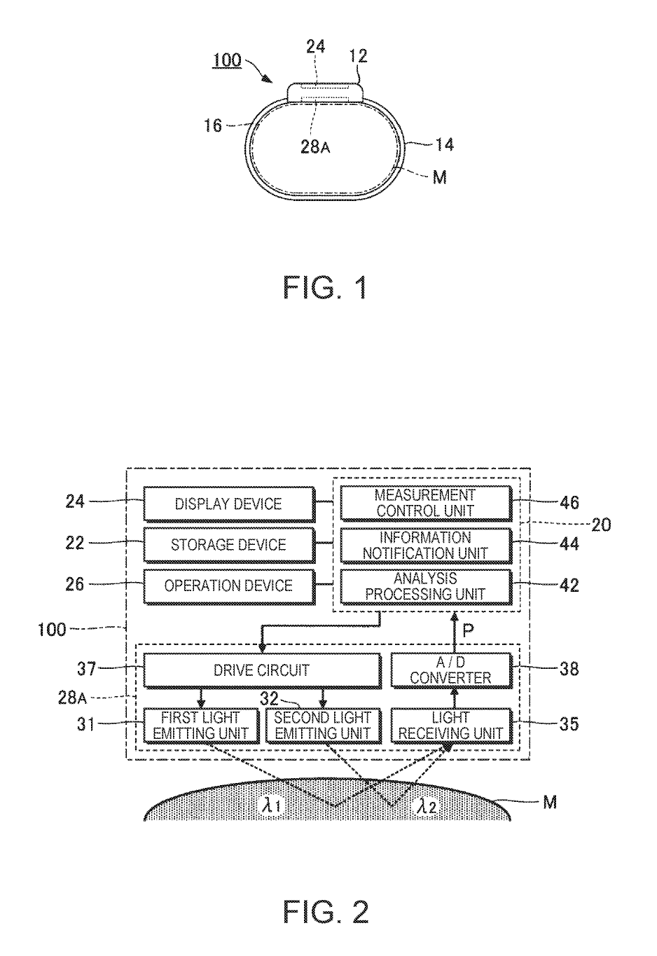

FIG. 1 is a side view of a measurement device 100 according to a first embodiment of the invention. The measurement device 100 according to the first embodiment is a biometric apparatus which noninvasively measures biological information of a subject, and is mounted on a site (hereinafter, referred to as a “measurement site”) M which is a measurement object in a body of the subject. The measurement device 100 according to the first embodiment is a wristwatch type portable device having a housing portion 12 and a belt 14, and can be attached to a wrist of the subject by winding the belt 14 of a strip shape around the wrist as an example of the measurement site M. The measurement device 100 according to the first embodiment comes into contact with the surface of the wrist of the subject. The first embodiment exemplifies a degree of oxygen saturation (SpO2) as the biological information. The degree of oxygen saturation indicates a proportion (%) of hemoglobin bonded to oxygen in the hemoglobin in blood of the subject, and is an index for evaluating a respiratory function of the subject.

FIG. 2 is a configuration diagram focusing on a function of the measurement device 100. As exemplified in FIG. 2, the measurement device 100 according to the first embodiment includes a control device 20, a storage device 22, a display device 24, an operation device 26, and a detection device 28A. The control device 20 and the storage device 22 are provided in the housing portion 12. As exemplified in FIG. 1, the display device 24 (for example, liquid crystal display panel) is provided on a surface (for example, a surface on a side opposite to the measurement site M) of the housing portion 12, and displays various images including measurement results in accordance with control of the control device 20. The operation device 26 is an input apparatus which receives inputs from a user (for example, a subject or a measurer). For example, a plurality of operation pieces that the user can operate, or a touch panel for detecting contact with a display surface of the display device 24 is suitably used as the operation device 26.

The detection device 28A of FIG. 2 is a sensor module which generates a detection signal P according to a state of the measurement site M, and is provided on, for example, a surface of the housing portion 12 which faces the measurement site M. As exemplified in FIG. 2, the detection device 28A according to the first embodiment includes a first light emitting unit 31, a second light emitting unit 32, a light receiving unit 35, a drive circuit 37, and an A/D converter 38.

Each of the first light emitting unit 31 and the second light emitting unit 32 is a light source which emits light onto the measurement site M. The first light emitting unit 31 emits light with a wavelength λ1 (example of a first wavelength) onto the measurement site M. The second light emitting unit 32 emits light with a wavelength λ2 (example of a second wavelength) different from the wavelength λ1 onto the measurement site M. In the first embodiment, a case where the first light emitting unit 31 emits red light (for example, λ1=600 nm to 800 nm) and the second light emitting unit 32 emits near infrared light (for example, λ2=800 nm to 1300 nm) is assumed for the sake of convenience. However, specific numerical values of the wavelength λ1 and the wavelength λ2 are not limited to the aforementioned examples. The drive circuit 37 makes each of the first light emitting unit 31 and the second light emitting unit 32 emit light by supplying a drive current.

Light which is emitted from each of the first light emitting unit 31 and the second light emitting unit 32 is incident on the measurement site M, repeats reflection and scattering in the measurement site M, emits on the housing portion 12 side, and reaches the light receiving unit 35. That is, the detection device 28A according to the first embodiment is an optical sensor of a reflection type in which first light emitting unit 31, second light emitting unit 32, and light receiving unit 35 are located on one side with respect to the measurement site M. The light receiving unit 35 generates the detection signal P according to a reception level of the light which reaches from the measurement site M. Since the amount of light absorbed by the blood in the blood vessel is different between time of expansion and time of contraction, the detection signal P which is generated by the light receiving unit 35 in accordance with the reception level of light from the measurement site M is a pulse wave signal including periodic variation components corresponding to pulsation components (volume pulse wave) of the artery in the measurement site M. The A/D converter 38 converts the analog detection signal P generated by the light receiving unit 35 into a digital signal.

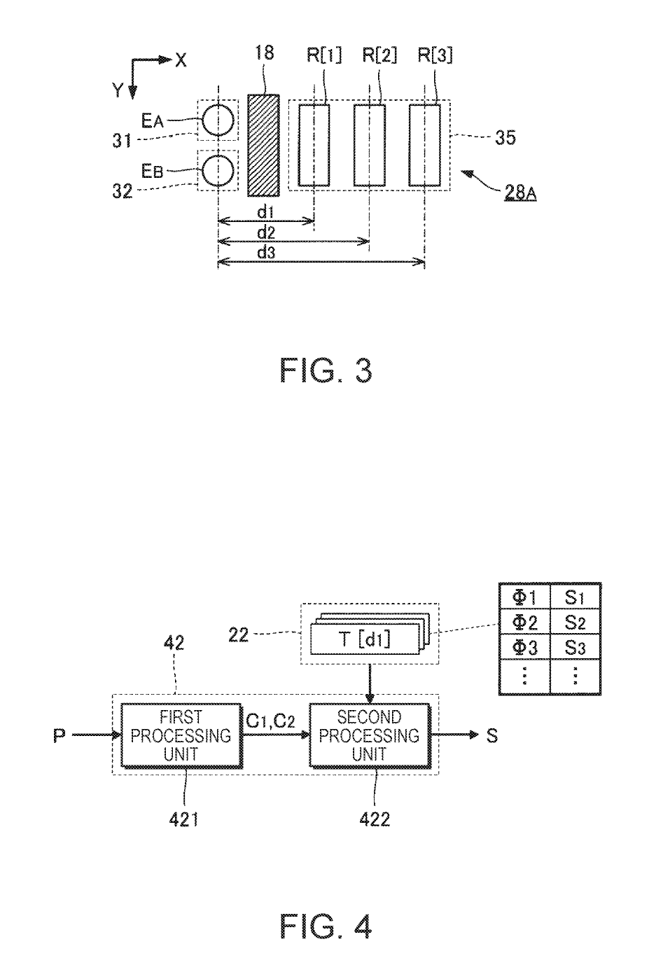

FIG. 3 is a plan view of the detection device 28A. An X-axis and a Y-axis which are orthogonal to each other are assumed on a surface of the housing portion 12 which faces the measurement site M. In the first embodiment, the first light emitting unit 31 is configured by one light emitting element EA and the second light emitting unit 32 is configured by one light emitting element EB, as exemplified in FIG. 3. Each of the light emitting element EA and the light emitting element EB is, for example, a light emitting diode (LED). The light emitting element EA and the light emitting element EB are arranged in the Y-axis direction.

As exemplified in FIG. 3, the light receiving unit 35 according to the first embodiment is configured by a plurality of light receiving elements R[n] (n=1, 2, 3). Each of the light receiving elements R[n] is, for example, a photo diode (PD) which receives light on a light receiving surface that faces the measurement site M. The plurality of light receiving elements R[n] are arranged in the X direction with a gap between those in a region on a positive side in the X direction as viewed from the first light emitting unit 31 and the second light emitting unit 32. Hence, distances between the light emitting element EA (light emission position by the first light emitting unit 31) and each (light reception position by the light receiving unit 35) of the plurality of light receiving elements R[n] are different from each other. In the same manner, distances between the light emitting element EB (light emission position by the second light emitting unit 32) and each of the plurality of light receiving elements R[n] are different from each other. In the aforementioned configuration, each of the plurality of light receiving elements R[n] can generate the detection signal P according to a light reception level, but in the first embodiment, the detection signal P generated by any one of the plurality of light receiving elements R [n] is selectively used for calculating the degree of oxygen saturation. A light blocking wall 18 for blocking light which directly travels from the first light emitting unit 31 or the second light emitting unit 32 on the light receiving unit 35 side is provided between the first light emitting unit 31, the second light emitting unit 32, and the light receiving unit 35.

The control device 20 of FIG. 2 is an operational processing device such as a central processing unit (CPU) or a field-programmable gate array (FPGA), and controls the entire measurement device 100. The storage device 22 is configured with, for example, a non-volatile semiconductor memory, and stores a program which is executed by the control device 20 and various types of data which are used for the control device 20. The control device 20 according to the first embodiment executes the program stored in the storage device 22, and performs a plurality of functions (an analysis processing unit 42, an information notification unit 44, and a measurement control unit 46) for specifying the degree of oxygen saturation of the subject. A configuration in which the functions of the control device 20 are distributed to a plurality of integrated circuit, or a configuration in which functions of a part of or the entire control device 20 are performed by a dedicated electronic circuit can also be adopted. In addition, the control device 20 and the storage device 22 are illustrated in FIG. 2 as different elements, but the control device 20 including the storage device 22 can also be realized by, for example, an application specific integrated circuit (ASIC).

The analysis processing unit 42 calculates the degree of oxygen saturation of a subject from the detection signal P generated by the detection device 28A (the light receiving unit 35). The information notification unit 44 notifies a user of the calculated degree of oxygen saturation calculated by the analysis processing unit 42. Specifically, the information notification unit 44 displays the degree of oxygen saturation on the display device 24 as measurement results. It is also possible for the information notification unit 44 to notify the user of the measurement results by voice output. The calculation of the degree of oxygen saturation performed by the analysis processing unit 42 and the notification of the degree of oxygen saturation which are performed by the information notification unit 44 are repeatedly performed, for example, for each predetermined time. It is also preferable to provide a configuration in which, in a case where the degree of oxygen saturation is varied into a numeric value out of a predetermined range, the information notification unit 44 notifies a user of a warning (possibility of disorder of respiratory function).

The analysis processing unit 42 according to the first embodiment can specify the degree of oxygen saturation by using a correlation between a variation ratio φ and the degree of oxygen saturation which are calculated from the detection signal P. The variation ratio φ is a ratio between a signal component ratio C1 and a signal component ratio C2 as represented by the following Expression (1). The signal component ratio C1 is an intensity ratio between variation components Q1 (AC) and normal components Q1 (DC) of the detection signal P when the first light emitting unit 31 emits light (red light) with the wavelength λ1. The signal component ratio C2 is an intensity ratio between variation components Q2 (AC) and normal components Q2 (DC) of the detection signal P when the second light emitting unit 32 emits light (near infrared light) with the wavelength λ2. The variation components Q1 (AC) and the variation components Q2 (AC) are components (pulse wave components) which periodically vary in conjunction with pulsation of the artery of the subject, and, for example, are extracted by a high pass filter as high frequency components of the detection signal P. The normal components Q1 (DC) and the normal components Q2 (DC) are components (DC components) which are normally maintained in time, and, for example, are extracted by a low pass filter as low frequency components of the detection signal P.

$$Φ = \frac{{C}_{2}}{{C}_{1}} = \frac{{Q}_{2 ( AC )} / {Q}_{2 ( DC )}}{{Q}_{1 ( AC )} / {Q}_{1 ( DC )}} \qquad ( 1 )$$

FIG. 4 is a configuration diagram of the analysis processing unit 42 according to the first embodiment. As exemplified in FIG. 4, the analysis processing unit 42 is configured to include a first processing unit 421 and a second processing unit 422. The first processing unit 421 calculates the signal component ratio C1 of Expression (1) and the signal component ratio C2 from the detection signal P. In the first embodiment, the drive circuit 37 of the detection device 28A makes the first light emitting unit 31 and the second light emitting unit 32 alternately emit light in time division during a cycle sufficiently shorter than a pulse rate. The first processing unit 421 calculates the variation components Q1 (AC) and the normal components Q1 (DC) from the detection signal P when the first light emitting unit 31 emits light, and calculates the intensity ratio between the normal components Q1 (DC) and the variation components Q1 (AC) as the signal component ratio C1. In the same manner, the first processing unit 421 calculates the variation components Q2 (AC) and the normal components Q2 (DC) from the detection signal P when the second light emitting unit 32 emits light, and calculates the intensity ratio between the normal components Q2 (DC) and the variation components Q2 (AC) as the signal component ratio C2. It is also possible to calculate the variation components Q1 (AC) and the variation components Q2 (AC), and the normal components Q1 (DC) and the normal components Q2 (DC), after a light reception level at the time of turning off (that is, when only ambient light such as sunlight or illumination light is received) of the first light emitting unit 31 and the second light emitting unit 32 is subtracted from the detection signal P.

The second processing unit 422 of FIG. 4 specifies the degree of oxygen saturation S from the signal component ratio C1 and the signal component ratio C2 which are calculated by the first processing unit 421. Specifically, the second processing unit 422 calculates the variation ratio φ by using calculation of Expression (1) in which the signal component ratio C1 and the signal component ratio C2 are applied, and specifies the degree of oxygen saturation S corresponding to the variation ratio φ by referring to a correlation table T in which correspondence between each numerical value of the variation ratio Φ and each numerical value of the degree of oxygen saturation S is registered. The specification of the degree of oxygen saturation S which uses the correlation table T will be described below.

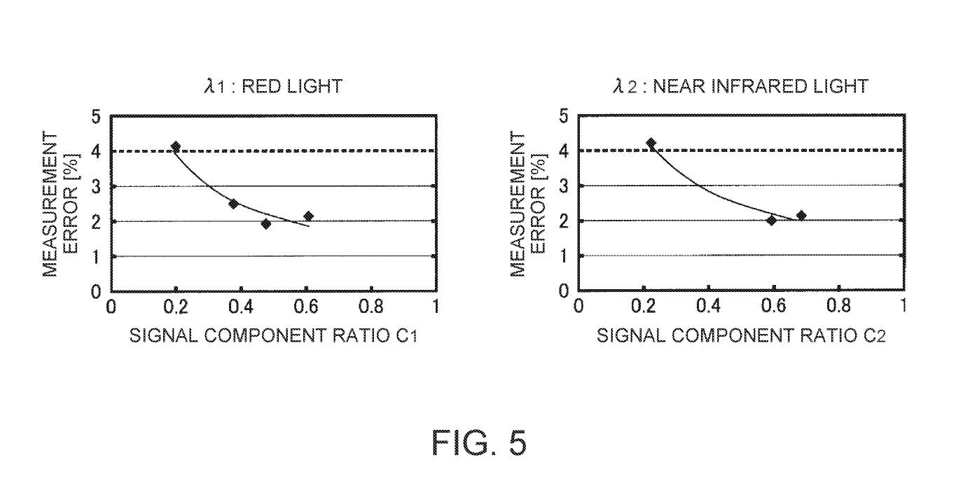

FIG. 5 is a graph illustrating a relationship between the signal component ratio C1 and the signal component ratio C2 and a measurement error [%] of the degree of oxygen saturation S. As can be seen from FIG. 5, there is a tendency that, the higher the signal component ratios C (C1, C2) are, the more the measurement error of the degree of oxygen saturation S decreases. That is, the larger the variation components Q1 (AC) is than the normal components Q1 (DC), or the larger the variation components Q2 (AC) is than the normal components Q2 (DC), the more the measurement error of the degree of oxygen saturation S decreases. For example, in order to decrease the measurement error to a value less than or equal to 4%, the signal component ratio C1 and the signal component ratio C2 which exceed 0.2 are required.

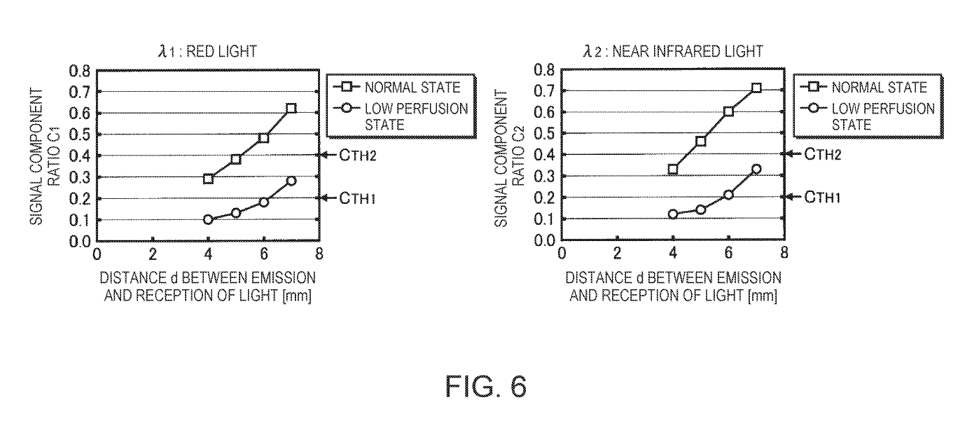

Meanwhile, FIG. 6 is a graph illustrating a relationship between a distance d between emission and reception of light and the signal component ratio C1 and the signal component ratio C2. The distance d between emission and reception of light means a distance between a light emission position and a light reception position. FIG. 6 illustrates relationships between the distance d between emission and reception of light and the signal component ratios C (C1, C2) together with respect to each of a normal perfusion state (hereinafter, referred to as a “normal state”) and a low perfusion state. As can be seen from FIG. 6, the distance d between emission and reception of light and the signal component ratio C correlate with each other. Specifically, there is a tendency that, the more the distance d between emission and reception of light increases, the higher the signal component ratio C is. Hence, in order to secure the signal component ratio C which can sufficiently reduce the measurement error of the degree of oxygen saturation S, the distance d between emission and reception of light need be increased. Meanwhile, in order for light which is emitted from the first light emitting unit 31 and the second light emitting unit 32 and passes through the inside of the measurement site M to reach the light reception position at sufficient intensity, the longer the distance d between emission and reception of light is, the more the amount of drive currents which are supplied to the first light emitting unit 31 and the second light emitting unit 32 are required to increase. As can be seen from the above description, there is a tendency that, the longer the distance d between emission and reception of light is, the more the measurement accuracy increases and the more power consumption increases.

In addition, as can be seen from FIG. 6, there is a tendency that the signal component ratio C with respect to the same distance d between emission and reception of light decreases in the low perfusion state, compared with the normal state. That is, in order to secure desired signal component ratio C in the low perfusion state, the distance d between emission and reception of light is required to increase, compared with the normal state.

As can be seen from the above description, in a case where the distance d between emission and reception of light is fixedly set in a relatively short dimension such that the desired signal component ratio C can be secured in the normal state, power consumption can be reduced, meanwhile there are problems that the sufficient signal component ratio C cannot be secured in the low perfusion state and the measurement error of the degree of oxygen saturation S decreases. Meanwhile, in a case where the distance d between emission and reception of light is fixedly set in a relatively long dimension such that the desired signal component ratio C can be secured in the low perfusion state, the degree of oxygen saturation S can be measured with a high accuracy, meanwhile there is a problem that power is consumed more than necessary in the normal state. In the first embodiment, it is possible to change the distance d between emission and reception of light in accordance with a perfusion state of a subject, based on the above tendency. Specifically, in the low perfusion state, the distance d between emission and reception of light is increased more than that in the normal state. The measurement control unit 46 of FIG. 2 variably controls the distance d between emission and reception of light.

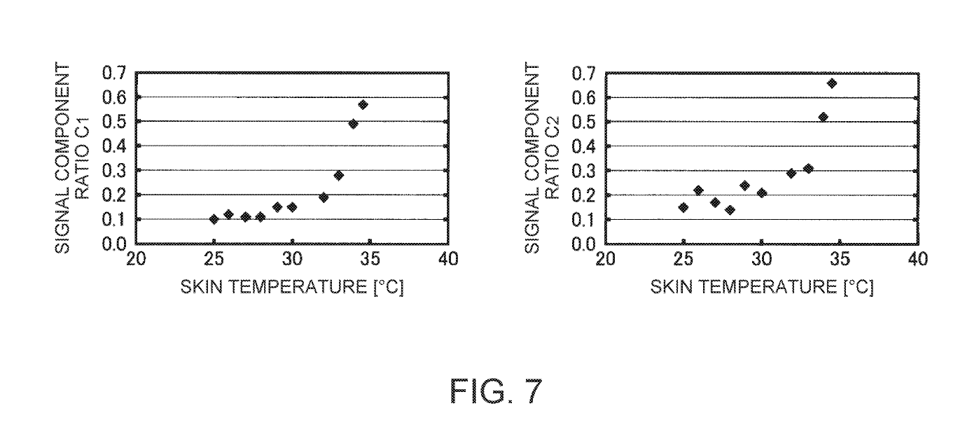

FIG. 7 is a graph illustrating a relationship between a skin temperature of the measurement site M and the signal component ratio C1 and the signal component ratio C2. Since the more the skin temperature decreases, the more perfusion of the subject decreases (that is, the amount of blood decreases), the skin temperature illustrated in a horizontal axis of FIG. 7 can be used as an index of a degree of the perfusion. As exemplified in FIG. 7, the skin temperature (degree of perfusion) of the measurement site M and the signal component ratio C correlate with each other. Specifically, a schematic tendency in which the more the perfusion decreases in conjunction with the skin temperature of the measurement site M, the more the signal component ratio C decreases, can be confirmed from FIG. 7. Considering the aforementioned tendency, the signal component ratio C which is calculated from the detection signal P by the analysis processing unit 42 can be used as the indexes of the degree of perfusion of the measurement site M. Specifically, it can be evaluated that the more the signal component ratio C decreases, the more the perfusion of the measurement site M decreases. Considering the aforementioned tendency, the measurement control unit 46 according to the first embodiment variably controls the distance d between emission and reception of light in accordance with the signal component ratio C1 or the signal component ratio C2.

As described above, the light receiving unit 35 according to the first embodiment is configured with a plurality of light receiving elements R[n]. The measurement control unit 46 can change the distance d between emission and reception of light by selecting the light receiving element R[n] which is actually used for specifying the degree of oxygen saturation S by the analysis processing unit 42 among the plurality of light receiving elements R[n] of the light receiving unit 35. Specifically, as exemplified in FIG. 3, as the measurement control unit 46 selects the light receiving element R[1], the distance d between emission and reception of light is set to a distance d1. In the same manner, as the measurement control unit 46 selects the light receiving element R[2], the distance d between emission and reception of light is set to a distance d2, and as the measurement control unit 46 selects the light receiving element R[3], the distance d between emission and reception of light is set to a distance d3.

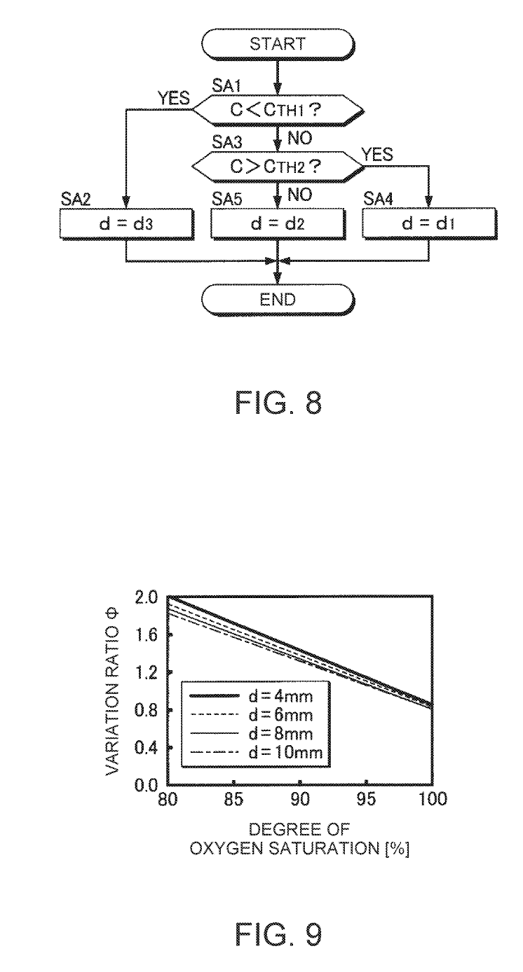

FIG. 8 is a flowchart of processing (processing of selecting the light receiving element R[n]) in which the measurement control unit 46 sets the distance d between emission and reception of light. For example, in a case where a user instructs measurement of the degree of oxygen saturation S by operating the operation device 26, the processing of FIG. 8 starts.

If the processing of FIG. 8 starts, the measurement control unit 46 determines whether or not the signal component ratio C (the signal component ratio C1 or the signal component ratio C2) is less than a threshold CTH1 (SA1). In a case where the signal component ratio C is less than the threshold CTH1 (SA1: YES), that is, in a case where it can be estimated that the measurement site M is in a low perfusion state, the measurement control unit 46 increases the distance d between emission and reception of light, compared with a case of the normal state (SA2). Specifically, the measurement control unit 46 selects the light receiving element R[3], thereby, setting the distance d between emission and reception of light to the distance d3. As described above, in order to decrease the measurement error of the degree of oxygen saturation S within 4%, it is necessary to maintain the signal component ratio C1 and the signal component ratio C2 in a value higher than or equal to 0.2%. Thus, the threshold CTH1 (example of a first threshold) is set to, for example, 0.2. As can be seen from FIG. 6, since the distance d between emission and reception of light is approximately 6 mm in a case where the signal component ratio C is 0.2, the distance d3 between emission and reception of light is set to a dimension longer than or equal to 6 mm (for example, 7 mm to 8 mm). As exemplified above, since the distance d between emission and reception of light is sufficiently secured, the sufficient signal component ratio C is secured even in the low perfusion state, and thereby, the degree of oxygen saturation S can be measured with high accuracy.

In a case where the signal component ratio C exceeds the threshold CTH1 (SA1: NO), the measurement control unit 46 determines whether or not the signal component ratio C (the signal component ratio C1 or the signal component ratio C2) exceeds a threshold CTH2 (SA3). The threshold CTH2 (example of a second threshold) is a numeric value which exceeds the threshold CTH1 (CTH2>CTH1), and is set to, for example, 0.4. In a case where the signal component ratio C exceeds the threshold CTH2, it is possible to secure the signal component ratio C with 0.2% or more, even if the distance d between emission and reception of light is shortened as compared with a case of the low perfusion state. Thus, in a case where the signal component ratio C exceeds the threshold CTH2 (SA3: YES), the measurement control unit 46 decreases the distance d between emission and reception of light as compared with a case of the low perfusion state (SA4). Specifically, the measurement control unit 46 selects the light receiving element R[1], thereby, setting the distance d between emission and reception of light to the distance d1.

Meanwhile, in a case where the signal component ratio C is a numeric value (CTH1≦C≦CTH2) between the threshold CTH1 and the threshold CTH2 (SA3: NO), the measurement control unit 46 selects the light receiving element R[2], thereby, setting the distance d between emission and reception of light to the distance d2 (SA5). The measurement control unit 46 can also variably control the amount of currents of a drive signal which is supplied to the first light emitting unit 31 and the second light emitting unit 32 by the drive circuit 37 in accordance with the signal component ratio C. Specifically, in a case where the signal component ratio C is less than the threshold CTH1 (low perfusion state), the drive signal is set to the amount of currents more than that in a case where the signal component ratio C exceeds the threshold CTH1. Meanwhile, in a case where the signal component ratio C exceeds the threshold CTH2, the drive signal is set to the amount of currents smaller than those in a case where the signal component ratio C is less than the threshold CTH2.

FIG. 9 is a graph exemplifying a relationship between the variation ratio φ which is calculated by Expression (1) described above and the degree of oxygen saturation S. FIG. 9 illustrates the relationships between the variation ratio φ and the degree of oxygen saturation S together, with respect to each of a plurality of cases where the distance d between emission and reception of light is changed. The aforementioned tendency in which the variation ratio φ and the degree of oxygen saturation S correlate with each other can be confirmed from FIG. 9. Specifically, there is a schematic tendency in which the larger the variation ratio φ is, the smaller the degree of oxygen saturation S is. In addition, the relationship between the variation ratio φ and the degree of oxygen saturation S changes depending on the distance d between emission and reception of light. Considering the aforementioned tendency, the storage device 22 according to the first embodiment stores a plurality of the correlation tables T corresponding to numeric values of the distance d between emission and reception of light different from each other, as exemplified in FIG. 4. The correlation table T corresponding to one arbitrary distance d between emission and reception of light is a data table in which correspondence (that is, the relationship exemplified in FIG. 9) of each numeric value between the variation ratio Φ and the degree of oxygen saturation S under the distance d between emission and reception of light is registered. The second processing unit 422 of the analysis processing unit 42 specifies, as a measurement result, the degree of oxygen saturation S corresponding to the variation ratio φ calculated by Expression (1), from the correlation table T corresponding to the distance d between emission and reception of light selected by the measurement control unit 46 among a plurality of the correlation tables T corresponding to the distance d between emission and reception of light different from each other.

As exemplified above, since the distance d between emission and reception of light varies in the first embodiment, it is possible to measure the degree of oxygen saturation S with high accuracy, even in a case where a state of the measurement site M changes (for example, low perfusion state). Particularly, in the first embodiment, the signal component ratio C which is used for specifying the degree of oxygen saturation S is used for selecting the distance d between emission and reception of light. Hence, there is an advantage that a configuration or processing for controlling the distance d between emission and reception of light is simplified as compared with a configuration in which the distance d between emission and reception of light is set in accordance with an index regardless of specifying the degree of oxygen saturation S.

In the first embodiment, the distance d between emission and reception of light increases in a case where the signal component ratio C is less than the threshold CTH1, and thus, it is possible to measure the degree of oxygen saturation S with high accuracy even in the low perfusion state. In addition, in a case where the signal component ratio C exceeds the threshold CTH2, the distance d between emission and reception of light decreases, and thus, it is possible to reduce the amount of currents necessary for making the first light emitting unit 31 and the second light emitting unit 32 emit light.

### Second Embodiment

A second embodiment of the invention will be described. In the respective aspects which will be exemplified below, the same symbols or reference numerals which are used for describing the first embodiment will be attached to the same elements as in the first embodiment, and detailed description thereof will be appropriately omitted.

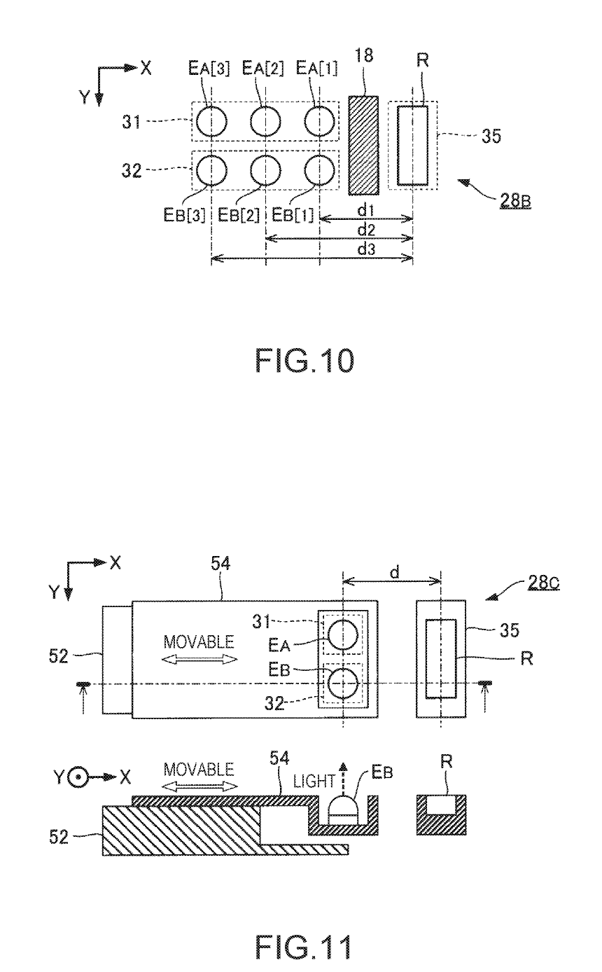

The measurement device 100 according to the second embodiment has a configuration in which the detection device 28A according to the first embodiment is replaced with a detection device 28B of FIG. 10. As exemplified in FIG. 10, a first light emitting unit 31 according to the second embodiment is configured with a plurality of the light emitting elements EA[n] which are arranged in the X direction, and a second light emitting unit 32 is configured with a plurality of the light emitting elements EB[n] which are arranged in the X direction (n=1, 2, 3). That is, distances between the plurality of the light emitting elements EA[n] and the light receiving unit 35 are different from each other, and distances between the plurality of the light emitting elements EB [n] and the light receiving unit 35 are different from each other. Meanwhile, the light receiving unit 35 is configured with one light receiving unit R. A measurement control unit 46 according to the second embodiment selects the light emitting element EA[n] and the light emitting element EB[n] which are driven by the drive circuit 37 (selectively emits light of any one of the plurality of the light emitting elements EA[n] and light of any one of the plurality of the light emitting elements EB[n]), thereby, variably controlling the distance d between emission and reception of light.

Specifically, in a case where the signal component ratio C is less than the threshold CTH1 (SA1: YES), the measurement control unit 46 selects the light emitting element EA[3] and the light emitting element EB[3] and the selected elements are driven by the drive circuit 37, and thereby, the distance d between emission and reception of light is set to the distance d3 (SA2). Meanwhile, in a case where the signal component ratio C exceeds the threshold CTH2 (SA3: YES), the measurement control unit 46 selects the light emitting element EA[l] and the light emitting element EB[l] and the selected elements are driven by the drive circuit 37, and thereby, the distance d between emission and reception of light is set to the distance d1 (SA4). In addition, in a case where the signal component ratio C is a numeric value between the threshold CTH1 and the threshold CTH2 (SA3: NO), the measurement control unit 46 selects the light emitting element EA[2] and the light emitting element EB[2] and the selected elements are driven by the drive circuit 37, and thereby, the distance d between emission and reception of light is set to the distance d2. As can be seen from the above example, the second embodiment also obtains the same effects as in the first embodiment.

It is also possible to employ both the configuration according to the first embodiment in which any one of the plurality of the light receiving elements R[n] is selected and the configuration according to the second embodiment in which any one of the plurality of the light emitting elements EA[n] and any one of the plurality of the light emitting elements EB[n] are selected. Specifically, the measurement control unit 46 selects any one of the plurality of light emitting elements EA[n] of the first light emitting unit 31 and any one of the plurality of light emitting elements EB [n] of the second light emitting unit 32, and selects any one of the plurality of light receiving elements R[n] of the light receiving unit 35. The distance d between emission and reception of light is variably set depending on a combination of the light emitting elements EA[n], the light emitting elements EB[n], and the light receiving elements R[n].

### Third Embodiment

In a third embodiment, the detection device 28A according to the first embodiment is replaced with a detection device 28C of FIG. 11. The detection device 28C includes a supporting body 52 and a moving object 54 in addition to the first light emitting unit 31, the second light emitting unit 32, and the light receiving unit 35. The first light emitting unit 31 is configured with one light emitting element EA, and the second light emitting unit 32 is configured with one light emitting element EB. In addition, the light receiving unit 35 is configured with one light receiving element R.

The first light emitting unit 31 and the second light emitting unit 32 are provided over the moving object 54. The supporting body 52 supports the moving object 54. Specifically, the moving object 54 is supported by the supporting body 52 in a movable state in the X direction. A user can move the moving object 54 to an arbitrary position in the X direction with respect to the supporting body 52 by appropriately operating the moving object 54. The first light emitting unit 31 and the second light emitting unit 32 move in the X direction together with the moving object 54. Meanwhile, a position of the light receiving unit 35 with respect to the supporting body 52 is fixed. As can be seen from the above description, the user can manually change the distance d between emission and reception of light.

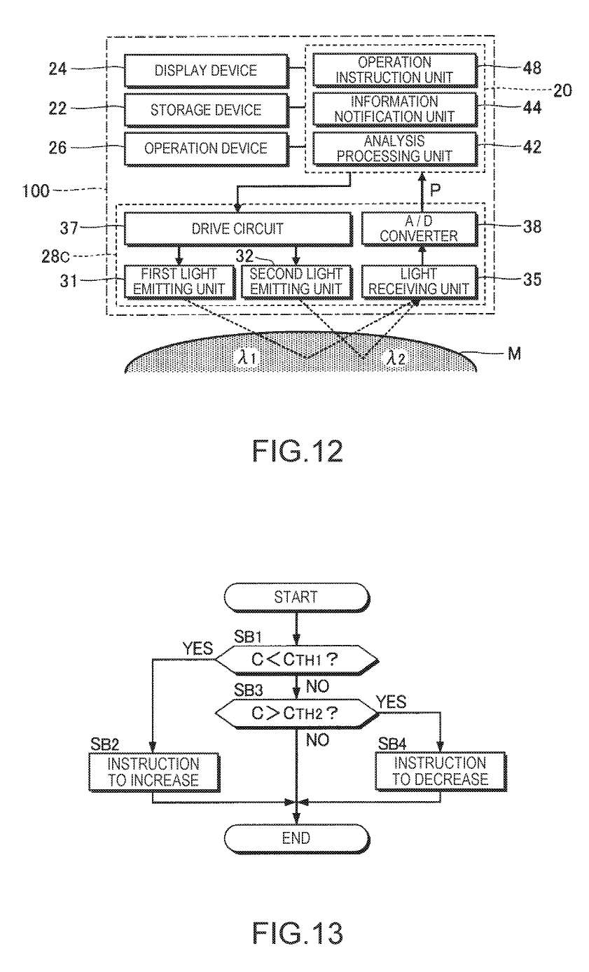

FIG. 12 is a configuration diagram focusing on a function of the measurement device 100 according to the third embodiment. As exemplified in FIG. 12, a control device 20 of the measurement device 100 according to the third embodiment functions as an operation instruction unit 48 in addition to the analysis processing unit 42 and the information notification unit 44 which are the same as in the first embodiment. That is, in the third embodiment, the measurement control unit 46 according to the first embodiment is replaced with the operation instruction unit 48. Processing in which the analysis processing unit 42 specifies the degree of oxygen saturation S from the detection signal P, and processing in which the information notification unit 44 notifies a user of measurement results are the same as in the first embodiment.

The operation instruction unit 48 notifies the user of instruction (hereinafter, referred to as “change instruction”) of a change of the distance d between emission and reception of light. Specifically, the operation instruction unit 48 displays the change instruction of the distance d between emission and reception of light according to the signal component ratios C (C1, C2) which are calculated by the analysis processing unit 42 on the display device 24. FIG. 13 is a flowchart of processing in which the operation instruction unit 48 notifies the user of the change instruction. For example, in a case where measurement of the degree of oxygen saturation S is instructed from the user, the processing of FIG. 13 starts.

If the processing of FIG. 13 starts, the operation instruction unit 48 determines whether or not the signal component ratio C (the signal component ratio C1 or the signal component ratio C2) is less than the threshold CTH1 (SB1). In a case where the signal component ratio C is less than the threshold CTH1 (SB1: YES), that is, in a case where it can be estimated that the measurement site M is in the low perfusion state, the operation instruction unit 48 notifies a user of the change instruction to increase the distance d between emission and reception of light (SB2). For example, a message such as “please move the light emission position away from the light reception position” is displayed on the display device as the change instruction. A user which completes confirmation of the change instruction increases the distance d between emission and reception of light by moving the moving object 54 to a negative side in the X direction. Since the distance d between emission and reception of light is sufficiently secured by the aforementioned procedure, the sufficient signal component ratio C is secured even in the low perfusion state, and thereby, the degree of oxygen saturation S can be measured with high accuracy. In a case where the distance d between emission and reception of light increases, the drive circuit 37 increases the amount of currents of the drive signal.

In a case where the signal component ratio C exceeds the threshold CTH1 (SB1: NO), the operation instruction unit 48 determines whether or not the signal component ratio C (the signal component ratio C1 or the signal component ratio C2) exceeds the threshold CTH2 (SB3). In the same manner as in the first embodiment, the threshold CTH2 is set to a numeric value (for example, 0.4) exceeding the threshold CTH1. In a case where the signal component ratio C exceeds the threshold CTH2 (SB3: YES), the operation instruction unit 48 notifies the user of the change instruction to decrease the distance d between emission and reception of light (SB4). For example, a message such as “please move the light emission position closer to the light reception position” is displayed on the display device 24 as the change instruction. A user which completes confirmation of the change instruction decreases the distance d between emission and reception of light by moving the moving object 54 in a positive side in the X direction. In a case where the distance d between emission and reception of light decreases, the drive circuit 37 decreases the amount of currents of the drive signal. Meanwhile, in a case where the signal component ratio C is a numeric value between the threshold CTH1 and the threshold CTH2 (SB3: NO), the operation instruction unit 48 does not notify the user of the change instruction. Hence, the distance d between emission and reception of light is maintained without being changed.

As exemplified above, since the distance d between emission and reception of light varies in the third embodiment, the degree of oxygen saturation S can be measured with high accuracy, even in the state of the measurement site M is changed (for example, the low perfusion state), in the same manner as in the first embodiment. Particularly, the signal component ratio C which is used for specifying the degree of oxygen saturation S is used for the change instruction with respect to the user, in the third embodiment. Hence, there is an advantage that a configuration or processing for notifying the user of the change instruction is simplified as compared with a configuration in which whether or not to perform the change instruction is determined in accordance with an index regardless of specifying the degree of oxygen saturation S.

In the third embodiment, in a case where the signal component ratio C is less than the threshold CTH1, change instruction to increase the distance d between emission and reception of light is given to the user, and thus, the degree of oxygen saturation S can be measured with high accuracy even in the low perfusion state. In addition, in a case where the signal component ratio C exceeds the threshold CTH2, the change instruction to decrease the distance d between emission and reception of light is given to the user, and thus, it is possible to reduce the amount of currents necessary for making the first light emitting unit 31 and the second light emitting unit 32 emit light.

### Fourth Embodiment

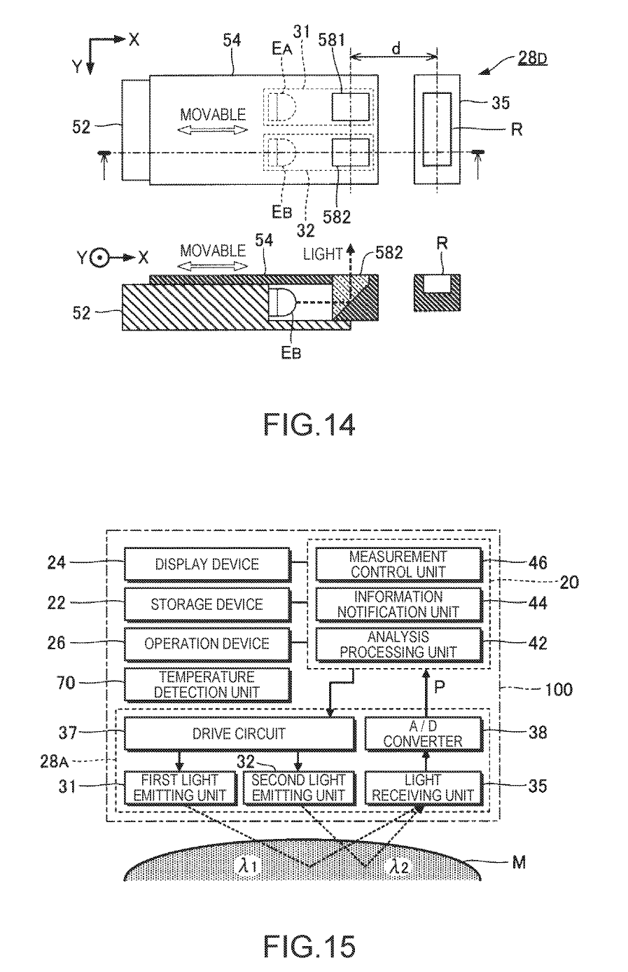

In a fourth embodiment, the detection device 28C according to the third embodiment is replaced with a detection device 28D of FIG. 14. The detection device 28D includes the first light emitting unit 31, the second light emitting unit 32, the light receiving unit 35, the supporting body 52, and the moving object 54. The first light emitting unit 31 includes the light emitting element EA and a light guiding unit 581. The light guiding unit 581 is an optical element which guides light emitted from the light emitting element EA on the measurement site M side. The second light emitting unit 32 includes the light emitting element EB and a light guiding unit 582. The light guiding unit 582 is an optical element which guides light emitted from the light emitting element EB on the measurement site M side. The light emitting element EA and the light emitting element EB are installed in the supporting body 52, and the light guiding unit 581 and the light guiding unit 582 are installed in the moving object 54. The light guiding unit 581 and the light guiding unit 582 can also be configured as one piece. The light receiving unit 35 is configured with one light receiving element R in the same manner as in the third embodiment.

In the same manner as in the third embodiment, the moving object 54 is supported by the supporting body 52 in a movable state in the X direction. A user can move the moving object 54 to an arbitrary position in the X direction with respect to the supporting body 52 by appropriately operating the moving object 54. While the positions of the light emitting element EA and the light emitting element EB are fixed, the light guiding unit 581, the light guiding unit 582, and the moving object 54 move together in the X direction. Meanwhile, a position of the light receiving unit 35 with respect to the supporting body 52 is fixed.

In the fourth embodiment, the light guiding unit 581 corresponds to the light emission position of the first light emitting unit 31, and the light guiding unit 582 corresponds to the light emission position of the second light emitting unit 32. Hence, the distance d between emission and reception of light according to the fourth embodiment is a distance between each of the light guiding unit 581 and the light guiding unit 582, and the light receiving unit 35. As can be seen from the above description, in the fourth embodiment, a user can manually change the distance d between emission and reception of light in the same manner as in the third embodiment.

An operation of the control device 20 (the analysis processing unit 42, the information notification unit 44, the operation instruction unit 48) is the same as in the third embodiment. For example, in a case where the signal component ratio C is less than the threshold CTH1, the operation instruction unit 48 instructs a user to increase the distance d between emission and reception of light, and in a case where the signal component ratio C exceeds the threshold CTH2, the operation instruction unit 48 instructs the user to decrease the distance d between emission and reception of light. Hence, the same effects as in the third embodiment can also be obtained in the fourth embodiment.

### Fifth Embodiment

In the first embodiment to the fourth embodiment, the signal component ratio C is used as the index of the degree of perfusion of the measurement site M, but the degree of perfusion of the measurement site M also depends on a skin temperature of the measurement site M or an ambient temperature (for example, ambient temperature in which the measurement device 100 is used). Specifically, the lower the skin temperature or the ambient temperature is, the more the perfusion of the measurement site M decreases. Considering the aforementioned circumstances, in the fifth embodiment and a sixth embodiment, a temperature (hereinafter, referred to as a “reference temperature”) such as the skin temperature or the ambient temperature which can affect the degree of perfusion of the measurement site M is used instead of the signal component ratio C as an index of the degree of perfusion of the measurement site M. Schematically, the fifth embodiment has a configuration in which the signal component ratio C of the first embodiment is replaced with the reference temperature, and the sixth embodiment has a configuration in which the signal component ratio C of the third embodiment is replaced with the reference temperature.

FIG. 15 is a configuration diagram of the measurement device 100 according to the fifth embodiment. As exemplified in FIG. 15, the measurement device 100 according to the fifth embodiment has a configuration in which a temperature detection unit 70 is added to the same elements as in the first embodiment. The temperature detection unit 70 is a temperature sensor which detects the skin temperature of the measurement site M or the ambient temperature around the measurement device 100 as a reference temperature KREF. The measurement control unit 46 according to the fifth embodiment variably controls the distance d between emission and reception of light in accordance with the reference temperature KREF detected by the temperature detection unit 70.

As described above, a tendency is assumed in which the lower the reference temperature KREF is, the more the perfusion of the measurement site M decreases. Considering the aforementioned tendency, the measurement control unit 46 of the fifth embodiment increases the distance d between emission and reception of light in a case where the reference temperature KREF is less than a predetermined threshold KTH1 (that is, in a case where it is assumed that the measurement site M is in the low perfusion state). As can be seen from FIG. 7, in a range in which the skin temperature is lower than 30° C., the signal component ratio C1 is suppressed to be less than or equal to 0.2, and as a result, it is difficult to sufficiently reduce the measurement error. Considering the aforementioned tendency, it is preferable to provide a configuration in which the distance d between emission and reception of light increases, in a case where the threshold KTH1 is set to 30° C. and the reference temperature KREF which is the skin temperature is lower than the threshold KTH1. Meanwhile, in a case where the reference temperature KREF exceeds a threshold KTH2 (KTH2>KTH1), the measurement control unit 46 decreases the distance d between emission and reception of light.

Also in the fifth embodiment, there is an advantage in which the degree of oxygen saturation S can be measured with high accuracy, even in a case where the state of the measurement site M changes (for example, the low perfusion state), in the same manner as in the first embodiment. The fifth embodiment is described on the basis of the first embodiment in the above description, but the configuration of the fifth embodiment in which the distance d between emission and reception of light is variably controlled in accordance with the reference temperature KREF detected by the temperature detection unit 70 can also be applied to the second embodiment which uses the detection device 28B of FIG. 10, in the same manner.

### Sixth Embodiment

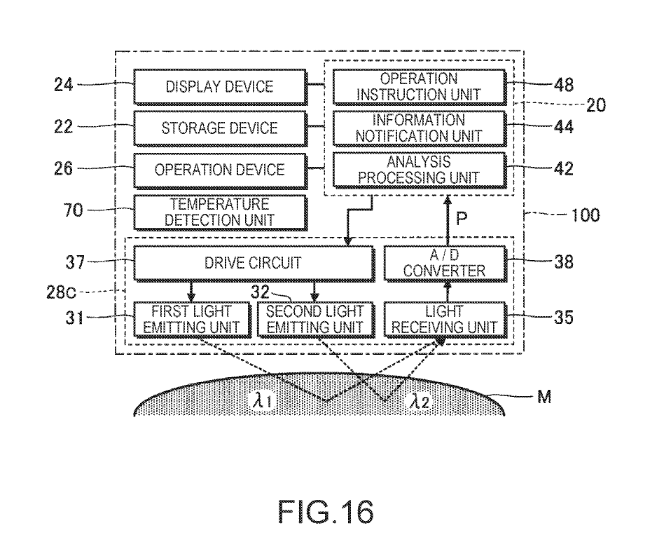

FIG. 16 is a configuration diagram of the measurement device 100 according to the sixth embodiment. As exemplified in FIG. 16, the measurement device 100 of the sixth embodiment has a configuration in which the temperature detection unit 70 is added to the same elements as in the third embodiment. In the same manner as in the fifth embodiment, the temperature detection unit 70 is a temperature sensor which detects the skin temperature of the measurement site M or the ambient temperature around the measurement device 100 as the reference temperature KREF.

The operation instruction unit 48 according to the sixth embodiment notifies a user of the change instruction of the distance d between emission and reception of light in accordance with the reference temperature KREF detected by the temperature detection unit 70. Specifically, in a case where the reference temperature KREF is lower than the predetermined threshold KTH1 (that is, in a case where it is assumed that the measurement site M is in the low perfusion state), the measurement control unit 46 notifies the user of the change instruction to increase the distance d between emission and reception of light. Meanwhile, in a case where the reference temperature KREF exceeds the threshold KTH2 (KTH2>KTH1), the measurement control unit 46 notifies the user of the change instruction to decrease the distance d between emission and reception of light.

Also in the sixth embodiment, there is an advantage in which the degree of oxygen saturation S can be measured with high accuracy, even in a case where the state of the measurement site M changes (for example, the low perfusion state), in the same manner as in the third embodiment. The sixth embodiment is described on the basis of the third embodiment in the above description, but the configuration of the sixth embodiment in which the user is notified of the change instruction in accordance with the reference temperature KREF detected by the temperature detection unit 70 can also be applied to the fourth embodiment which uses the detection device 28D of FIG. 14, in the same manner.

### Modification Example

Each embodiment exemplified above can be modified in various types. Specific modification aspects will be exemplified below. It is also possible to appropriately combine two or more aspects which are arbitrarily selected from the below example.

(1) In the respective embodiments described above, the threshold CTH1 and the threshold CTH2 are set as fixed values, but it is also possible to use variable values which vary depending on predetermined conditions as the threshold CTH1 and the threshold CTH2. For example, it is preferable to provide a configuration in which the threshold CTH1 or the threshold CTH2 is variably set in accordance with an operation mode of the measurement device 100.

(2) In the respective embodiments described above, the analysis processing unit 42 specifies the degree of oxygen saturation S by using the correlation table T, but a method of specifying the degree of oxygen saturation S is not limited to the above example. For example, the analysis processing unit 42 can also perform arithmetic of the degree of oxygen saturation S by applying the variation ratio φ to an arithmetic expression derived by using the Lambert-Beer's law.

(3) In the third embodiment and the fourth embodiment, the change instruction is displayed on the display device 24, but a method of notifying a user of the change instruction is not limited to the above example. For example, a configuration in which the user is notified of the change instruction by lighting the light emitting element, a configuration in which the user is notified of the change instruction by voice output, or a configuration in which the user is notified of the change instruction by providing the user with vibration of a predetermined pattern can also be employed.

(4) In the third embodiment and the fourth embodiment, a case is exemplified in which a user manually moves the moving object 54 in which the first light emitting unit 31 and the second light emitting unit 32 are installed, but a configuration for moving the moving object 54 is not limited to the above example. For example, the measurement control unit 46 can also move the moving object 54 by operating a drive mechanism including an actuator such as a motor. Specifically, the measurement control unit 46 increases the distance d between emission and reception of light by moving the moving object 54 to a negative side in the X direction, in a case where the signal component ratios C (C1, C2) are less than the threshold CTH1. Meanwhile, in a case where the signal component ratio C exceeds the threshold CTH2, the measurement control unit 46 moves the moving object 54 to a positive side in the X direction, thereby, decreasing the distance d between emission and reception of light. In the third embodiment (FIG. 11) and the fourth embodiment (FIG. 14), a configuration in which the first light emitting unit 31 and the second light emitting unit 32 moves with respect to the light receiving unit is exemplified, but it is preferable to provide a configuration in which the light receiving unit 35 can move with respect to the first light emitting unit 31 and the second light emitting unit 32, instead of the aforementioned configuration (or together with the aforementioned configuration).

(5) In the respective embodiments described above, the analysis processing unit 42 which is mounted in the measurement device 100 specifies the degree of oxygen saturation S, but a device other than the measurement device 100 can also specify the degree of oxygen saturation S. For example, it is assumed that a configuration in which a terminal device (for example, a mobile phone or a smart phone) which can communicate with the measurement device 100 specifies and displays the degree of oxygen saturation S is provided. Specifically, the terminal device receives the detection signal P from the measurement device 100, and calculates the degree of oxygen saturation S from the detection signal P by using the method exemplified in the respective embodiments described above. As can be seen from the above description, the analysis processing unit 42 and the information notification unit 44 can be omitted from the measurement device 100. The measurement control unit 46 can also be installed in the terminal device. As described above, it is also possible to employ a configuration (for example, a configuration in which each element is realized by an application that the terminal device executes) in which at least one of the analysis processing unit 42, the information notification unit 44, and the measurement control unit 46 is installed in the terminal device. In addition, it is also possible to install one of or both the storage device 22 and the operation device 26 in the terminal device.

(6) In the respective embodiments described above, the measurement device 100 which can be mounted on a wrist of a subject is exemplified, but, a specific form (wear position) of the measurement device 100 is arbitrary. The measurement device 100 having arbitrary form, such as a patch type that can be attached to the body of the subject, an earring type that can be mounted on an ear of the subject, a finger-mounted type (for example, a nail clipper) that can be mounted on a fingertip of the subject, or a head mount type which can be mounted on a head of the subject, can be employed. However, since there is a possibility that everyday life is disruptive in a state where the measurement device 100 of, for example, the finger-mounted type is mounted, it is particularly preferable to provide the measurement device 100 of the respective embodiments described above which can be mounted on the wrist of the subject from the viewpoint of measuring the oxygen saturation S at any time without disrupting everyday life. The measurement device 100 of a form which is mounted (for example, externally attached) on electronic apparatuses of various types such as a wristwatch can also be realized.

(7) In the respective embodiments described above, the degree of oxygen saturation S is measured, but types of biological information are not limited to the aforementioned examples. For example, it is also possible to employ a configuration in which pulsation, blood flow velocity or blood pressure is measured as the biological information, and a configuration in which various blood component concentrations, such as blood glucose concentration, hemoglobin concentration, blood oxygen concentration, or neutral fat concentration are measured as the biological information. A laser irradiator that emits a coherent laser beams which is emitted through resonance generated by a resonator in a narrowband is preferably used as the first light emitting unit 31 and the second light emitting unit 32, in the configuration in which blood flow velocity is measured as the biological information.

The entire disclosure of Japanese Patent Application No. 2016-141677 is hereby incorporated herein by reference.

What is claimed is:

1. A measurement device comprising:
a first light emitting unit that emits light with a first wavelength onto a measurement site;
a second light emitting unit that emits light with a second wavelength different from the first wavelength onto the measurement site;
a light receiving unit that receives light which passes through an inside of the measurement site and generates a detection signal; and
an analysis processing unit that calculates a degree of oxygen saturation from the detection signal,
wherein distances between light emission positions by each of the first light emitting unit and the second light emitting unit and a light reception position by the light receiving unit are variable.

2. The measurement device according to claim 1, wherein the analysis processing unit includes
a first processing unit that calculates a signal component ratio between normal components and variation components of the detection signal, and
a second processing unit that specifies the degree of oxygen saturation from the signal component ratio.

3. The measurement device according to claim 2, further comprising:
a measurement control unit that increases a distance between the light emission position and the light reception position, in a case where the signal component ratio is less than a first threshold.

4. The measurement device according to claim 3, wherein the measurement control unit decreases the distance between the light emission position and the light reception position, in a case where the signal component ratio exceeds a second threshold exceeding the first threshold.

5. The measurement device according to claim 3,
wherein the light receiving unit includes a plurality of light receiving elements whose distances from the first light emitting unit and the second light emitting unit are different from each other, and
wherein the measurement control unit changes a distance between the light emission position and the light reception position by selecting any one of the plurality of light receiving elements.

6. The measurement device according to claim 3,
wherein each of the first light emitting unit and the second light emitting unit includes a plurality of light emitting elements whose distances from the light receiving unit are different from each other, and
wherein the measurement control unit changes the distance between the light emission position and the light reception position by selectively emitting any one of the plurality of light emitting elements, in each of the first light emitting unit and the second light emitting unit.

7. The measurement device according to claim 2,
wherein the distance between the light emission position and the light reception position is changeable by an operation of a user, and
wherein the measurement device further comprises an operation instruction unit that notifies the user of instruction to increase the distance between the light emission position and the light reception position, in a case where the signal component ratio is less than the first threshold.

8. The measurement device according to claim 7, wherein the operation instruction unit notifies the user of instruction to decrease the distance between the light emission position and the light reception position, in a case where the signal component ratio exceeds a second threshold exceeding the first threshold.

9. The measurement device according to claim 1, further comprising:
a temperature detection unit that detects a skin temperature or an ambient temperature of the measurement site as a reference temperature; and
a measurement control unit that increases the distance between the light emission position and the light reception position, in a case where the reference temperature is lower than a threshold.

10. The measurement device according to claim 1, further comprising:
a temperature detection unit that detects a skin temperature or an ambient temperature of the measurement site as a reference temperature; and
an operation instruction unit that notifies a user of instruction to increase the distance between the light emission position and the light reception position, in a case where the reference temperature is lower than a threshold.

11. A detection device that generates a detection signal which is used for measuring a degree of oxygen saturation, the detection device comprising:
a first light emitting unit that emits light with a first wavelength onto a measurement site;
a second light emitting unit that emits light with a second wavelength different from the first wavelength onto the measurement site; and
a light receiving unit that receives light which passes through an inside of the measurement site and generates a detection signal,
wherein distances between light emission positions by each of the first light emitting unit and the second light emitting unit and a light reception position by the light receiving unit are variable.
